(19) United States
(12) Patent Application Publication
(10) Pub. No.: US 2015/0218513 A1
(43) Pub. Date: Aug. 6, 2015

(54) HEPATOCYTE PREPARATIONS

(71) Applicant: LIFE TECHNOLOGIES CORPORATION, Carlsbad, CA (US)

(72) Inventors: Mark POWERS, New Market, MD (US); Rafal WITEK, Burlington, NC (US); Kirsten AMARAL, Cary, NC (US)

(21) Appl. No.: 14/331,039

(22) Filed: Jul. 14, 2014

Related U.S. Application Data
(63) Continuation of application No. 13/813,913, filed on Feb. 1, 2013, now abandoned.
(60) Provisional application No. 61/438,942, filed on Feb. 2, 2011.
(60) Provisional application No. 61/370,429, filed on Aug. 3, 2010.

(51) Int. Cl. C12N 5/071

(52) U.S. Cl. CPC C12N 5/067

(57) ABSTRACT

Methods for preparing a variety of cell (e.g., hepatocyte) preparations and preparations so prepared are described. Uses of such preparations are described. In one embodiment, centrifugal elutriation is used to separate hepatocytes with preferred characteristics. Such hepatocyte preparations may be used for cryopreservation, multiple cryopreservations, in vitro assays, plating and other methods for which preparations of hepatocytes are useful.

### CROSS REFERENCE TO RELATED APPLICATIONS

This application is a continuation of U.S. application Ser. No. 13/813,913, filed Feb. 3, 2013, now abandoned, which is a national stage application filed under 35 U.S.C. §371(c) of International Application No. PCT/US2011/046485, filed Aug. 3, 2011, which claims the benefit under 35 U.S.C. 119(e) of U.S. Provisional Patent Application No. 61/438,942, filed Feb. 2, 2011 and of U.S. Provisional Patent Application No. 61/370,429, filed Aug. 3, 2010, all of which are hereby expressly incorporated by reference in their entirety as though fully set forth herein.

### TECHNICAL FIELD

The present invention relates to cell preparations, a method for obtaining such preparations, and a method for using such cell preparations. In addition, the invention concerns methods of processing preparations of such cells so as to enhance their viability and quality and further allow their single or repeated cryopreservation and thawing while retaining substantial viability and quality. The invention also concerns preparations of cells that have been so prepared.

### BACKGROUND

The liver is the primary site of metabolism for most drugs. Hepatocytes are parenchymal liver cells and play a key role in the detoxification, modification and excretion of natural and synthetic compounds in the systemic circulation (Ponsoda et al. (2004) Altern Lab Anim 32(2): 101-110). Because of their importance in xenobiotic metabolism, hepatocytes are of great interest to the research community and pharmaceutical industry. Over the past decade, primary hepatocytes have become a standard in vitro tool to evaluate hepatic drug metabolism, cytochrome P450 induction, and drug interactions affecting hepatic metabolism.

Protocols have been developed for the preparation and culture of isolated hepatocytes (LeCluyse et al. (2005) Methods Mol Biol 290:207-229). This ability to culture and/or isolate hepatocytes have made such preparations an attractive model system for the study of liver functions (Chesne et al. (1993) Hepatology 18(2):406-414; Guillouzo (1998) Environ Health Perspect 106 (Suppl 2):511-532; Ponsoda et al. (2004) Altern Lab Anim 32(2): 101-110; Gomez-Lechon et al. (2004) Curr Drug Metab 5(5):443-462; Lemaigre et al. (2004) Curr Opin Genet Dev 14(5):582-590; Nanji (2004) Clin Liver Dis 8(3):559-574; Hewitt et al. (2004) Hum Exp Toxicol 23(6):307-316). However, the use of density based techniques may have limitations for some desired applications.

A limiting factor in the development of such model systems and to the development of bioartificial livers has been, for example, (1) the erratic source and limited availability of hepatocytes (especially human hepatocytes); and (2) the presence of cellular debris, cell clumping, and other artifacts present in hepatocyte preparations.

Fresh hepatocytes are obtainable from liver resections or non-transplantable livers of multi-organ donors and the supply of such tissue is inconsistent and often geographically inconvenient in light of the limited functional lifespan of liver tissue (Lloyd et al. (2003) Cell Tissue Bank 4:3-15; Smrzova et al. (2001) Acta Veterinaria Brunensis 70:141-147).

Even with fresh tissue, isolation techniques result in preparations exhibiting less than 100% viability. Such preparations commonly contain cellular debris, clumped cells, dead cells, and other artifacts of the purification process. These “contaminants” may further damage the preparation if the preparation is stored or cryopreserved for later use. In any event, such contaminants can affect variability, metabolic function, assay capacity and yield.

Hepatocytes can also be derived from in vitro differentiation of pluripotent cells, such as embryonic stem cells or induced pluripotent stem cells (iPSCs). Recently, methods have been developed whereby hepatocytes derived in vitro from differentiation of pluripotent cells can be produced on an industrial scale. In such large scale processes, the hepatocytes are isolated, for example, from 1 liter or more of culture media or solutions. Such preparations commonly contain dead cells, clumped cells, cellular debris, as well as cells of different types or other artifacts of the differentiation, culturing, and/or purification process.

Hepatocyte storage conditions, including the use of cyropreservation, have been developed that allow hepatocytes to be maintained over time with their cellular functions preserved (see, Lloyd et al. (2003) Cell Tissue Bank 4:3-15; Loretz et al. (1989) Xenobiotica 19(5):489-498; Shaddock et al. (1993) Cell Biol Toxicol 9(4):345-357; Novicki et al. (1982) In Vitro 18(4):393-399; Zaleski et al. (1993) Biochem Pharmacol 46(1):111-116). Typically, such cryopreservation measures comprise storage in liquid nitrogen (−196° C.) or in frozen nitrogen gas (−150° C.). The ability to recover viable thawed cells has been found to depend on multiple factors such as the rate of freezing, the concentration of hepatocytes, the type of cryoprotectant employed, and the final cooling temperature. Cell concentrations of 10⁶-10⁷ cells/ml have been typically employed. The isolated hepatocytes may or may not be incubated in suspension for a period (e.g., 4-48 hours) to allow them to recover from the isolation process. Thereafter, a cryoprotectant (such as glycerol, DMSO, polyvinylpyrrolodine, or dextran) is added, and the hepatocytes are frozen. The art has developed various freezing procedures, all designed to minimize or prevent the occurrence of intracellular ice. The freezing rates typically vary from −0.05° C./min to −50° C./min (see, Lloyd et al. (2003) Cell Tissue Bank 4:3-15).

Another major problem affecting the use of both fresh and cryopreserved hepatocytes is the variation of liver enzyme expression that is observed in tissue from different donors (Li, A. P. et al. (1999) Chem Biol Interact 121(1):117-123; Li, A. P. et al. (1999) Chem Biol Interact 121(1):17-35). One solution to this sample variation involves pooling samples from different sources (Shibata et al. (2002) Drug Metab Dispos 30:892-896). In addition, liver enzyme expression may vary based on the point of origin in the liver from which the hepatocytes were originally derived or other parameters associated with the donor (i.e. obesity, smoking, etc.). Accordingly, there may be use for preparations of hepatocytes derived from a particular region of the liver or pools of hepatocytes derived from a particular region of the liver from multiple individuals or any combination derived from donors with specific parameters or characteristics (i.e. high Body Mass Index (BMI)).

While the development of cryopreservation methods for the storage of hepatocytes has significantly facilitated the availability of human hepatocytes, the isolation techniques and the ability to separate viable and non-viable hepatocytes both before and after thawing (for example, before use or before pooling and re-freezing) have depended on density centrifugation (whether by gradient or non-gradient). Density centrifugation is a common technique for separating viable from non-viable cells in hepatocyte preparations. Examples of this include gradient density centrifugation (i.e. discontinuous, continuous, layered etc.), for example, with PERCOLL® or other similar compositions. Such a technique is described in U.S. Pat. No. 7,604,929. However, because this technique separates cells by density alone, it is unable, for example, to separate clumped cells from individual cells (a clump of like cells having the same density as a single cell of the same type). Clumped cells have been shown to exhibit more variable metabolic activity and may be less healthy than unclumped hepatocytes. In addition, it may not separate less viable cells from more viable cells (less viable cells being not yet dead, capable of exhibiting the same or similar density to viable cells, but having other distortions of shape etc. that indicate their poor prognosis). Further, because the cells are purified in a density medium, the final preparation will be contaminated by, for example, the density component. This may impair the use of the cells in a variety of downstream steps including, for example, FACS analysis or other techniques for using and/or manipulating cells. In general, hepatocytes prepared using the technique of elutriation have not been well evaluated, particularly when those hepatocytes have been cryopreserved two or more times prior to use or when those hepatocytes have been prepared from in vitro differentiated hepatoctyes.

Periportal hepatocytes around the afferent vessels and perivenous hepatocytes around the efferent vessels of the liver acinus exhibit different metabolic capacities and subcellular structures. In that respect, the liver acinus is described as comprising different zones. Oxidative energy metabolism, gluconeogenesis, urea synthesis, bile formation and protective metabolism occur primarily in the periportal zone; while glycolysis (arising from liponeogenesis), glutamine synthesis and xenobiotic metabolism occur primarily in the perivenous zone. Zones develop gradually, and is dependent on hepatic circulation, energy substrates, maturity of the host and environmental factors including diet and disease. In addition, gradients of oxygen which exist across the liver lobules, hormones, metabolites and other factors result in heterogeneous gene expression within the liver acinus. See, e.g., Katz (1992) J Nutr 122: 843-849. However, density fractionation techniques are unable to separate hepatocytes with the fine precision necessary to separate hepatocytes based on their zone of origin or other physical characteristics dependent thereon.

Thus, despite all prior advances, a need remains for processes that would enable the availability of hepatocytes for medical research and other purposes. A need further exists for a stable and reproducible source of human hepatocytes. The present invention permits the production and availability of extremely clean hepatocyte preparations that may be used while fresh, cryopreserved, or repeatedly cryopreserved without unacceptable loss of viability. The invention thus permits multiple hepatocyte samples to be pooled to produce pooled hepatocyte preparations, especially pooled cryopreserved human hepatocyte preparations.

### SUMMARY

The present invention has an object to provide cell(s) and cell preparations and methods for preparing, for example, hepatocyte(s) and hepatocyte preparations. The present invention further provides a method of using centrifugal elutriation to purify, for example, hepatocyte cells and hepatocyte preparations produced by this method. The invention further provides a method of using plating to manipulate hepatocyte preparations in desired ways. Hepatocyte preparations prepared by the methods described herein may have various advantages and may, for example, be distinguished using fluorescence-activated cell sorting (FACS) or flow cytometry analysis from preparations made using prior methods. For example, the invention relates to preparations of hepatocytes that exhibit greater percentages of “good” hepatocytes (as shown, for example, in flow cytometric analysis) as compared to hepatocyte preparations existing in the prior art.

The inventors have surprisingly found that the technique of centrifugal elutriation can be applied to preparations of isolated hepatocytes in such a way as to enhance many desired properties of such hepatocyte preparations. In that respect, the inventors have found that centrifugal elutriation can be applied as an improved substitute for the density centrifugation or density gradient centrifugation steps as practiced by the prior art. Methods employing centrifugal elutriation include those in which hepatocytes are cryopreserved once, twice, or more than twice, for example, as part of a pooling process. Alternatively, centrifugal elutriation can be used in combination with such density based fractionation steps, for example, in method employing one or more cryopreservation steps.

Hepatocyte preparations made by the described techniques may, for example, be useful in cell suspension or plating and used accordingly.

In one aspect, a method is provided for producing a clean preparation of isolated hepatocytes from a less clean preparation of isolated hepatocytes. The method comprises subjecting the less clean preparation of isolated hepatocytes to centrifugal elutriation and collecting a fraction with the clean preparation of hepatocytes, wherein the clean preparation of hepatocytes demonstrates at least one of the following attributes as compared to the less clean preparation of isolated hepatocytes: less cellular debris; less cell clusters; less membrane blebbing; increased viability; increased metabolic activity; increased ability to withstand cryopreservation; or increased yield of a targeted cell population.

In another aspect, provided is a clean preparation of hepatocytes produced by any of the provided methods.

In another aspect, provided is a method of investigating in vitro drug metabolism. The method comprises incubating the clean preparation of hepatocytes in the presence of a xenobiotic, and determining the metabolic fate of the xenobiotic, or the affect of the xenobiotic on the hepatocytes or on an enzyme or metabolic activity thereof.

In another aspect, methods are provided for separating hepatocytes from different Zones. In one embodiment, a method is provided for separating a Zone 1 hepatocyte from a Zone 2 or Zone 3 hepatocyte comprising elutriating a hepatocyte composition comprising at least one Zone 1 hepatocyte and at least one hepatocyte selected from a Zone 2 hepatocyte and a Zone 3 hepatocyte. In another embodiment, a method is provided for separating a Zone 2 hepatocyte from a Zone 1 or Zone 3 hepatocyte comprising elutriating a hepatocyte composition comprising at least one Zone 2 hepatocyte and at least one hepatocyte selected from a Zone 1 hepatocyte and a Zone 3 hepatocyte. In another embodiment, a method is provided for separating a Zone 3 hepatocyte from a Zone 1 or Zone 2 hepatocyte comprising elutriating a hepatocyte composition comprising at least one Zone 3 hepatocyte and at least one hepatocyte selected from a Zone 1 hepatocyte and a Zone 2 hepatocyte.

In another aspect, provided is a preparation of hepatocytes, characterized by at least one of the following: less than 10% of the total number of hepatocytes are present in identifiable clumps; less than 10% of the total mass of the preparation is cellular debris; less than 10% of the hepatocytes in the preparation exhibit membrane blebbing; less than 10% of the hepatocytes exhibit the characteristics of Zone 1; less than 10% of the hepatocytes exhibit the characteristics of Zone 2; or less than 10% of the hepatocytes exhibit the characteristics of Zone 3.

In some embodiments, the hepatocytes are collected at less than 1000 rpm during centrifugal elutriation.

In some embodiments, the hepatocytes are derived from liver tissue. In other embodiments, the hepatocytes are non-tissue derived hepatocytes, such as hepatocytes derived from induced pluripotent stem cells differentiated in vitro.

In some embodiments, the less clean preparation is from a single donor. In other embodiments, the less clean preparation is from more than one donor resulting in a pooled preparation of hepatocytes.

In some embodiments, the less clean preparation has been cryopreserved at least once. In some embodiments, the less clean preparation has been cryopreserved at least twice.

In some embodiments, the less clean preparation comprises a mammalian hepatocyte, such as a hepatocyte selected from the group consisting of a human hepatocyte, a porcine hepatocyte, a simian hepatocyte, a canine hepatocyte, a feline hepatocyte, a bovine hepatocyte, an equine hepatocyte, an ovine hepatocyte and a rodent hepatocyte.

In some embodiments, a pooled preparations of the hepatocytes comprises hepatocytes selected with respect to at least one metabolic activity, such as a metabolic activity is selected from the group consisting of coumarin 7-hydroxylase (COUM), dextromethorphan O-demethylase (DEX), 7-ethoxycourmarin O-deethylase (ECOD), activities responsible for the phase II metabolism of 7-hydroxycoumarin (7-HCG and 7-HCS), mephenytoin 4-hydroxylase (MEPH), testosterone 6 (beta)-hydroxylase (TEST), tolbutamide 4-hydroxylase (TOLB), phenacetin O-deethylase (PHEN), chloroxazone 6-hydroxylase (CZX), paclitaxel hydroxylase, and bupropion hydroxylase.

In some embodiments, the clean preparation exhibits at least about 70% viability. In some embodiments, the clean preparation exhibits at least about 80% viability.

In some embodiments, the hepatocyte preparation is substantially free of a density gradient medium, such as the hepatocyte preparation is PERCOLL®-free.

In some embodiments, the hepatocytes are: substantially derived from a donor with BMI>30, substantially derived from a donor with BMI>40, or substantially derived from a donor with BMI>50.

In some embodiments, the hepatocytes are: substantially derived from a donor with BMI≦30, substantially derived from a donor with BMI≦40, or substantially derived from a donor with BMI≦50.

In another aspect, provided is a preparation of hepatocytes that have been subjected to two rounds of cryopreservation with an intervening elutriation step, wherein the hepatocytes are further characterized by a flow cytometric analysis score of at least 20.

In another aspect, provided is a preparation of hepatocytes that have been subjected to at least two rounds of cryopreservation with at least one intervening elutriation step. In some embodiments, the hepatocytes are subjected to only two rounds of cryopreservation and only one intervening elutriation step.

In another aspect, provided is a preparation of hepatocytes having an enhanced ability to attach to a culture surface. In some embodiments, at least about 50% of the preparation of hepatocytes is able to attach to the surface after incubation. In some embodiments, at least about 70% of the preparation of hepatocytes is able to attach to the surface after inc In some embodiments, at least about 90% of the preparation of hepatocytes is able to attach to the surface after incubation.

In another aspect, provided is a multi-well culture plate comprising a preparation of hepatocytes wherein at least one well comprises a nearly confluent monolayer of the hepatocytes. In one embodiment, the multi-well plate comprises at least 96 wells. In one embodiment, the multi-well plate comprises at least 384 wells and at least one well is seeded with about 15,000 to about 20,000 hepatocytes.

### BRIEF DESCRIPTION OF THE DRAWINGS

FIG. 1A, FIG. 1B, FIG. 1C and FIG. 1D are representative phase contrast images of elutriated hepatocytes. FIG. 1A shows debris and nonparenchymal cells (NPC) collected at 1400 rpm. FIG. 1B shows dead hepatocytes collected at 1400-1000 rpm. FIG. 1C shows live hepatocytes collected at 1000-400 rpm. FIG. 1D shows clumps and aggregates collected at 400-0 rpm. All centrifugation speeds were performed at constant 50 mL/minute flow rate.

FIG. 2A and FIG. 2B are representative phase contrast images illustrating the ability to separate fractions of hepatocytes based on their Zone(s) of origin. FIG. 2A is a 1000-800 rpm collection representing a smaller size population of hepatocytes. This size is predominately isolated from Zones 1 or 2. FIG. 2B is a 800-600 rpm collection representing a larger size population of hepatocytes. This size is predominately isolated from Zones 2 or 3.

FIG. 3 is a bar graph depicting increased viability of elutriated hepatocytes when compared from post thaw to final count prior to the second cryopreservation.

FIG. 4 is a bar graph depicting the percent recovery of hepatocytes by elutriation. Recovery in 1000-400 g collection range. Hu 8081, Hu 8062, and Hu 0562/8017 represent samples with low starting viability (under 70%). Hu 0619 and Hu 8082-2 represent samples with high starting viability (over 70%). Historical data indicates that recovery from PERCOLL® centrifugation would be ˜50%.

FIG. 5A and FIG. 5B illustrate elutriation vs. standard isolation. FIG. 5A shows a representative phase contrast image of elutriated hepatocyte preparation according to the invention. The sample shows little evidence of debris, NPC, cell clusters or membrane blebbing. FIG. 5B shows a representative phase contrast image of standard isolation technique using PERCOLL® centrifugation. The sample shows evidence of debris (1), NPC (1), cell clusters (2) and membrane blebbing (3). Methodology of elutriation can also be applied to fresh hepatocytes with similar results.

FIG. 6A and FIG. 6B are representative phase contrast images illustrating zonality of liver function. FIG. 6A shows Zone 1 periportal hepatocytes. They are mostly single nucleated hepatocytes and usually contain little or no lipid vesicles. They are high oxygen, oxidative metabolism, glucogenesis, and ureagenesis. FIG. 6B shows Zone 3 represents mostly bi-nucleated hepatocytes, can contain lipid vesicles. These are low oxygen, useful for glycolysis, lipogenesis, xenobiotic metabolism.

FIG. 7 illustrates the structure of a liver lobule with indication of Zones.

FIG. 8A, FIG. 8B, FIG. 8C, FIG. 8D, FIG. 8E, and FIG. 8F show flow cytometric analysis of hepatocyte preparations prepared by isolating hepatocytes from liver tissue, freezing, thawing, subjecting to either PERCOLL® centrifugation (FIG. 8B, FIG. 8D and FIG. 8F) or elutriation (FIG. 8A, FIG. 8C and FIG. 8E), cryopreserving and thawing. In essence, samples in FIG. 8A, FIG. 8C and FIG. 8E are prepared according to one embodiment of the instant invention while samples in FIG. 8B, FIG. 8D and FIG. 8F are prepared according to techniques (i.e. cryopreservation and purification techniques) described in the prior art.

### DETAILED DESCRIPTION

Centrifugal elutriation (including counterflow centrifugal elutriation, counterstreaming centrifugal elutriation and countercurrent centrifugal elutriation) protocols have been developed for the separation of cells. See, for example, Brouwer et al. “Centrifugation separations of mammalian cells.” Preparative Centrifugation, pp. 271-314, Ed. Rickwood, Oxford, IRL Press, 1992; Bruyninckx et al. (1990) “Characterization of the efficiency of a cell separation process by the extent of elimination of a contaminating cell type.” Anal. Biochem. 191:144-155; “Centrifugal elutriation of living cells. An annotated bibliography.” Applications Data DS-534. Palo Alto, Beckman Instruments, Inc., 1990; Diamond (1991) “Separation and enrichment of cell populations by centrifugal elutriation.” Methods 2:173-182; Dorin, “Developing Elutriation Protocols” (Technical Information: High Speed Centrifugation), © 1994 Beckman Instruments, Inc.; Figdor “Separation of subpopulations from heterogeneous human monocytes.” Cell Separation 4:295-308, Ed. Pretlow et al. Orlando, Academic Press, 1987; Figdor et al. (1981) “Isolation of large numbers of highly purified lymphocytes and monocytes with a modified centrifugal elutriation technique.” J Immunol Methods 40:275-288; Figdor et al. (1983) “Theory and practice of centrifugal elutriation (CE). Factors influencing the separation of human blood cells.” Cell Biophys. 5:105-118; Kauffman et al. (1990) “Isolation of cell cycle fractions by counterflow centrifugal elutriation.” Anal Biochem 191:41-46; Keng “High-capacity separation of homogeneous cell subpopulations by centrifugal elutriation.” Cell Separation Science and Technology, pp. 103-112. Ed. Kompala et al., Washington, American Chemical Society, 1991. [ACS Symposium Series 464]; Mason et al. (1985) “Application of the Beckman JE6-B Elutriator System® in the isolation of human monocyte subpopulations.” Scand. J. Haematol. 34:5-8; Meistrich “Experimental factors involved in separation by centrifugal elutriation.” Cell Separation 2:33-61, Ed. Pretlow et al. New York, Academic Press, 1983; Pretlow et al. (1979) “Centrifugal elutriation (counterstreaming centrifugation) of cells.” Cell Biophys 1:195-210; Sanders et al. (1989) “Cell separation by elutriation: major and minor cell types from complex tissues.” Methods in Enzymology 171:482-497; Sanderson et al. “Cell separations by counterflow centrifugation.” Methods in Cell Biology, 15:1-14, Ed. Prescott, N.Y., Academic Press, 1977; Sanderson et al. (1976) “Design principles for a counterflow centrifugation cell separation chamber. Appendix: a derivation of the equation of motion of a particle under combined centrifugal and hydrodynamic fields.” Anal Biochem 71:615-622; Sharpe, Methods of Cell Separation, Chap. 5: Centrifugal elutriation, pp. 91-106. Amsterdam, Elsevier, 1988. [Lab. Techn. Biochem. Mol. Biol., v. 18]; and Wahl et al. “Isolation of monocytes by counterflow centrifugal isolation.” Current Protocols in Immunology, 1:7.6.3-7.6.8. Ed. J. E. Coligan et al. New York, J. Wiley, 1991.

However, the applications of centrifugal elutriation to isolated hepatocyte preparations and/or the qualities and characteristics of hepatocyte preparations made using centrifugal elutriation (and as described herein) have not been described.

The centrifugal elutriation method provided herein results in cell preparations enriched for hepatocytes over nonparenchymal cells (NPC) of the liver, including, for example, sinusoidal endothelial cells, Kupffer cells, and hepatic stellate cells. In addition to enriching for hepatocytes, one of skill in the art will appreciate that these methods may be utilized in enriching for subpopulations of NPC, including but not limited to, Kupffer cells, sinusoidal endothelial cells, and hepatic stellate cells. Accordingly, in one embodiment, a method is provided for producing a clean preparation of isolated NPC subpopulation from a preparation of liver cells wherein the preparation of liver cells is subjected to centrifugal elutriation and a fraction including the desired NPC subpopulation is collected.

Cell preparations that result from the use of at least one centrifugal elutriation step may be enriched for hepatocytes, usually at least about 50% hepatocytes by number, more usually at least about 80% hepatocytes by number, and preferably at least 90% hepatocytes by number.

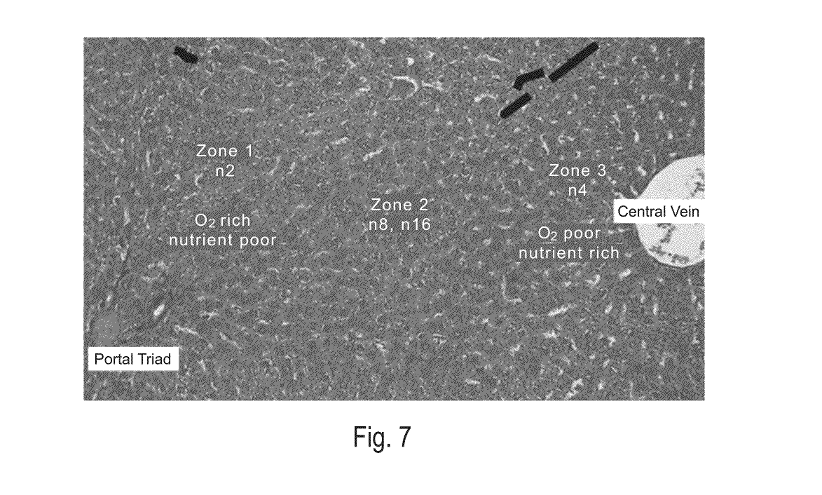

The liver is divided histologically into lobules. The center of the lobule is the central vein. At the periphery of the lobule are portal triads. Functionally, the liver can be divided into three Zones, based upon oxygen supply (FIG. 7). Zone 1 encircles the portal tracts where the oxygenated blood from hepatic arteries enters and is, therefore, oxygen rich. Zone 3 is located around central veins, where oxygenation is poor. Zone 2 is located in between. The hepatocytes in Zone 3 are sometimes bi-nucleated and exhibit enhanced metabolic activities as compared to hepatocytes from Zones 1 and 2. Zone 1 hepatocytes are often smaller in size than hepatocytes from Zones 2 and 3. Zone 2 hepatocytes have been found to show enhanced properties in induction assays.

Induction is an adaptive response process in the liver of mammals, including humans, whereby the expression of drug/xenobiotic clearance genes (e.g. metabolism, transporters) is upregulated to facilitate elimination of the foreign chemical. Assays for this are performed per FDA regulations by drug developers in cultures of primary human hepatocytes to predict in human-derived culture models whether induction is likely in humans. If induction is not observed in vitro under ‘ideal’ conditions with high quality cultures of primary human hepatocytes, drug labels are populated with text stating induction was not observed in vitro. However, if induction is observed in vitro, clinical trials are performed to determine the likelihood of this drug-drug interaction mechanism.

Put another way, liver lobules can be defined as functional units. The classic hepatic lobule is roughly hexagonal in shape, with its borders defined by an imaginary line connecting the surrounding portal spaces. The center is the central vein. The hepatic acinus is roughly diamond shaped with its borders defined by an imaginary line connecting the central veins of two neighboring classical lobules with two adjacent periportal spaces. It is functionally defined as the region that is irrigated by the terminal distributing branch of the portal vein. The cells in the hepatic acinus are subdivided into three Zones 1-3 based on their proximity to the distributing veins. The incoming blood passes first through Zone 1 and continues into Zone 2 and 3. The gradient for substances coming in with the blood (nutrients, oxygen, toxic substances) thus defines functional differences among the hepatocytes in the different Zones. The portal lobule is roughly triangular with its borders defined by an imaginary line connecting the three central veins that are adjacent to one portal space in the center. It is functionally defined as the area from which bile flows to one branch of the bile duct.

It is commonly accepted that interlot variability among primary human hepatocyte preparations is simply donor variability since it is well-established that genetic and environmental factors lead to substantial interindividual difference with respect to drug metabolism and transport, two primary focal points of primary human hepatocyte applications. However, what is not widely known by applications scientists is that all primary human hepatocytes are equivalent but in the unique microenvironments of Zones 1, 2, and 3 of the lobule, hepatocytes adapt to the unique conditions (e.g. oxygen levels) to specialize in functionality (e.g. CYP3A4 expression higher in Zone 3 than Zones 1 and 2).

When primary human hepatocytes are detached from the tissue during standard perfusion processes, cells from the various Zones are mixed together. Depending on factors such as diet (e.g. intracellular fat content), cell health, genetics, etc., these mixed cells during standard density gradient processing can vary greatly in density from patient to patient, and depending which cells survive this process and pass through the density gradient, this can lead to unique distributions of the subpopulations of zonal cells from patient to patient. In addition, standard processing using a fixed gradient limits the ability to select the appropriate cells from a given donor preparation.

The centrifugal elutriation fractionation methods provided herein allow isolationists to collect all the cells and pool the appropriate subpopulations with minimal damage. The method offers the ability to focus the isolation on the cells of interest (e.g., a zonal subpopulation) for particular applications. Accordingly, in certain embodiments, primary hepatocyte preparations produced by such methods have reduced interlot variability as compared to lots prepared by other purification methods. Such hepatocyte lots provide users a more consistent product from lot-to-lot and one that is more representative of true donor variability.

Accordingly, one embodiment of the invention is the use of centrifugal elutriation to separate hepatocytes based on their Zone of origin. Preparations rich in hepatocytes derived from one or more of the various Zones may exhibit characteristics that make them more useful or better suited for use in a particular assay. One of skill in the art would understand which assays would best be suited for such preparations. In another embodiment, pools of hepatocytes from more than one donor can be prepared that are similarly rich in hepatocytes from a particular Zone.

Hepatocytes demonstrate metabolic heterogeneity across the different Zones. See, e.g., Katz (1992) J Nutr 122: 843-849. Table 1 lists examples of Zonal metabolic heterogeneity of hepatocytes.

TABLE 1

| Metabolic Function - | |
|---|---|
| Predominant Zone | Exemplary activities |
| Oxidative metabolism - Zone 1 | Succinate dehydrogenase; malate |
| | dehydrogenase; cytochrome c oxidase |
| Glucogenesis - Zone 1 | 6-phosphatase; fructose |
| | 1,6-bisphosphatase; |
| | phosphoenolpyruvate carboxykinase; |
| | alanine aminotransferase; |
| | lactate dehydrogenase |
| Glycolysis - Zone 3 | Glucokinase; pyruvate kinase |
| Amino acid degradation - Zone 1 | Amino acid transferases; |
| Ureagenesis - Zone I and Zone 2 | Ornithine carbamoyltransferase |
| Ammonia detoxification - Zone 3 | Glutamine synthetase |
| Lipogenesis - Zone 3 | Fatty acid synthesis; very low density |
| | lipoprotein esterification and |
| | formation |
| Bile formation, uptake, | Canalicular ATPse; |
| and secretion - Zone 1 | hydroxymethylglutaryl-CoA |
| | reductase |
| Xenobiotic metabolism - Zone 3 | Cytochrome P-450 isoenzymes; |
| | NADP-reducing enzymes; UDP- |
| | glucuronosyltransferase |

Accordingly, preparations rich in hepatocytes of a particular Zone may be particularly suited for use in assays directed to a particular metabolic function or enzyme activity. For example, oxidative energy metabolism, glucogenesis, urea synthesis, bile formation and protective metabolism are performed mainly in hepatocytes of Zone 1. Also for example, glycolysis linked lipogenesis, glutamine synthesis, and xenobiotic metabolism are performed predominantly in hepatocytes of Zone 3. Zone 2 hepatocytes generally have lower baselines with respect to metabolism, (e.g., xenobiotic metabolism) and differentiated phenotypes.

Accordingly, in some instances, hepatocyte preparations rich in hepatocytes from Zone 1 would be particularly suited for assays determining the effect of a test agent or test condition on oxidative energy metabolism, for example, on enzyme activities involved in the citric acid cycle and respiratory chain reactions. In certain instances, hepatocyte preparations rich in hepatocytes from Zone 3 would be particularly suited for assays determining the effect of a test agent or test condition on xenobiotic metabolism.

An activity of cytochrome P-450 enzymes of Zone 3 hepatocytes forms toxic electrophilic metabolites, such as bromobenzene. Although such electrophiles are detoxified by combination with glutathione catalyzed by glutathione-S-transferase, the level of glutathione in hepatocytes decreases from Zone 1 to Zone 3 and Zone 3 hepatocytes demonstrate a Zone-specific toxicity, for example, of halogenated organic compounds. Accordingly, in certain instances, hepatocyte preparations rich in hepatocytes from Zone 3 would be particularly suited for assays assessing the effect of a test agent or test condition on hepatocyte toxicity, for example toxicity associated with xenobiotics or halogenated organic compounds.

In one embodiment, a preparation is rich in hepatocytes from a particular Zone if the percentage of hepatocytes from that Zone is greater than would (1) be otherwise expected; or (2) naturally occur in the liver.

In one embodiment, provided is a hepatocyte preparation enriched in Zone 3 hepatocytes. In some embodiments, provided is a hepatocyte preparation enriched in Zone 3 hepatocytes with high basal cytochrome P450 metabolism, e.g., CYP3A4.

In another embodiment, provided is a hepatocyte preparation enriched in Zone 2 hepatocytes. In certain embodiments, an enriched preparation of Zone 2 hepatocytes would be well suited for induction assessments. Induction of primary hepatocytes allows for measurement of their total metabolic capacity.

In one embodiment, provided is a hepatocyte preparation enriched in Zone 1 hepatocytes. In certain embodiments, an enriched preparation of Zone 1 hepatocytes would be well suited for cell proliferation models or assays.

Zonal expression patterns of certain transporters have been reported. In certain embodiments, hepatocyte preparation enriched in a particular Zone would be well suited for transporter functionality assays.

The variability seen in hepatocytes from individual to individual can be based on both genetic and other factors. For example, diet, weight, smoking, drug and alcohol use, viral infections etc. can affect hepatocyte quality and characteristics. For example, hepatocytes isolated from a donor with a Body Mass Index (BMI) of more than 30 often exhibit fat accumulation within individual hepatocytes. This fat accumulation may be in vacuoles within the hepatocytes. The presence of fat can change the size or buoyancy of the hepatocytes. Similarly, other factors may alter such qualities or characteristics of a hepatocyte. The use of density or density gradient fractionation in the isolation of such hepatocytes may be problematic. For example, buoyant hepatocytes may not separate at the same density as less buoyant hepatocytes.

Accordingly, in one embodiment of the invention, centrifugal elutriation is used to isolate such hepatocytes. For example, centrifugal elutriation may be used to make a hepatocyte preparation that is consistently or substantially comprised of hepatocytes comprising high levels of fat. Alternatively, centrifugal elutriation can be used to isolate hepatocytes that exhibit changes to their size or shape that might not be easily isolated using a density based isolation step.

In some embodiments, the hepatocytes of a preparation are substantially derived from a donor with BMI≦30. In certain embodiments, greater than 50% of the hepatocytes are derived from a donor with BMI≦30, greater than 70% of the hepatocytes are derived from a donor with BMI≦30, greater than 75% of the hepatocytes are derived from a donor with BMI≦30, greater than 80% of the hepatocytes are derived from a donor with BMI≦30, or greater than 90% of the hepatocytes are derived from a donor with BMI≦30.

In some embodiments, the hepatocytes of a preparation are substantially derived from a donor with BMI≦40. In certain embodiments, greater than 70% of the hepatocytes are derived from a donor with BMI≦40 or greater than 90% of the hepatocytes are derived from a donor with BMI≦40. In some embodiments, the hepatocytes of a preparation are substantially derived from a donor with BMI≦50.

Elutriation speeds may be adjusted to collect live fractions of hepatocytes. For example, using a Beckman Coulter JE-5.0 elutriator rotor, a less clean hepatocyte preparation is loaded at a rotor speed of 1400 rpm. The live fraction of hepatocytes are generally collected at an elutriation speed from 1000 to 400 rpm. Hepatocytes may be collected at intermediate fractions to separate different size and density hepatocytes based on zonal distribution or fat content, or collected as one large fraction. The speed of 1000 rpm to 800 rpm will elute the small size hepatocytes, for example, predominantly Zone 1 hepatocytes. The speed of 800 rpm to 600 rpm will elute larger size hepatocytes and some fatty cells, for example, predominantly Zone 2 cells with some overlap with Zone 1 and Zone 3 cells. The speed of 600 rpm to 400 rpm will elute large bi-nucleated cells and fatty cells, for example predominantly Zone 3 cells. These speeds may be further adjusted to increase purity or homogeneity of cells from a particular zone. The speed of 400 rpm to 0 rpm will elute the fraction which contains the aggregated and clumps of hepatocytes.

Hepatocyte preparations may be (but need not be) plated at any stage in the process. Preferably, if hepatocytes are to be plated they will be plated prior to elutriation. Preferably, such plating is done by adding medium comprising hepatocytes to a plate. In one embodiment, the plate will have collagen or other substrate attachment material(s). In one embodiment the medium comprising hepatocytes will be incubated for 5, 10, 15, 30 or 45 minutes. In another embodiment, the incubation will be for 1 hour, 2 hours or more. In one embodiment the incubation will be at four degrees centigrade. In another embodiment, the incubation will be performed at or near room temperature. In another embodiment, the incubation will performed above room temperature, for example, at 37° C. Following incubation, the medium may be decanted for further processing of the hepatocytes. In one embodiment, the incubation is performed at the appropriate temperature and for the appropriate time to allow faster attaching components to attach to the plate while the plated viable hepatocytes remain in suspension. In one embodiment, the faster attaching components comprise non-viable hepatocytes. In one embodiment, the faster attaching components include contaminating (i.e. non-hepatocyte) cells such as, for example, Kupffer cells.

Flow cytometry may be used to analyze and distinguish hepatocyte preparations. Wigg et al. (2003) Anal Biochem 317:19-25. For example, hepatocyte preparations may be characterized by side scatter and forward scatter. Based on this technique, the percentage of desirable hepatocytes in the preparation can be calculated.

In one embodiment, hepatocyte preparations of the instant invention demonstrate an increase in the percentage of desirable hepatocytes as indicated by flow cytometric analysis as compared to equivalent preparations made using prior art techniques. In a preferred embodiment, the inventive preparation shows a 10% improvement over prior art techniques. In further preferred embodiments, the improvement is greater than 20%, greater than 30%, greater than 40%, greater than 50%, greater than 75%, or greater than 100%.

As used herein, a “clean” preparation of hepatocytes or the “cleanliness” of a hepatocyte preparation refers to characteristics, attributes or qualities of a hepatocyte preparation including, for example, purity, cell viability, cell integrity, cell activity, ability to withstand cryopreservation, homogeneity or enrichment with regard to, for example, zonal origin, cell type, or cell buoyancy, and limited level of cellular debris or cell clusters.

Density gradient centrifugation is commonly used in cell preparation methods for separating viable from non-viable cells, including hepatocyte preparation methods. Examples of this include gradient density centrifugation (i.e. discontinuous, continuous, layered etc.), for example, with gradient media such as PERCOLL® or other similar compositions. PERCOLL® has been noted as generally non-toxic to cells, however certain phenomena have been associated with cell exposure to PERCOLL® including for example, PERCOLL® particle ingestion by cells, depression of motility, inhibition of phagocytic activity, and reduced adherence to plastic (see, for example, Wakefield et al. (1982) Biochem J. 202:795-797). The inventors have surprisingly found that the presence of PERCOLL® in a hepatocyte preparation can affect viability determinations and may impact the use of these preparations in metabolic studies. Thus, exposure of cells to PERCOLL® prior to use in an assay, for example, may affect cell viability, morphology, activity, as well as variability between cell lots and runs of the assay. A hepatocyte preparation made without PERCOLL® eliminates any artifact that may result from exposure to PERCOLL® or the presence of residual PERCOLL® in the cell preparation.

The methods of the instant invention provide cell preparations without the use of a density gradient medium, for example, without PERCOLL®. Accordingly, in one embodiment, a method is provided for isolating and purifying hepatocytes without the use of a density gradient medium. In another embodiment, provided is a hepatocyte preparation substantially free of density gradient medium, such as free of residual density gradient medium. In certain embodiments, a PERCOLL®-free hepatocyte preparation is provided.

In one embodiment, the invention provides a method which for purifying hepatocytes wherein hepatocytes that have been previously cryopreserved and thawed are subjected to elutriation, cryopreserved (i.e. cryopreserved for what would effectively be a second (or more) time) and stored. In one embodiment, the hepatocytes are plated prior to elutriation. In one embodiment, the hepatocyte preparation produced by this method is thawed. In one embodiment, the thawed preparation is used in in vitro assays, for example, in drug metabolism assays.

In one embodiment the hepatocyte preparation of this method comprises hepatocytes originally derived from more than one source.

In one embodiment, the source is mammalian. In one embodiment, the source is human, pig, monkey, dog, mouse or rat.

In some embodiments, the hepatocytes are derived from liver tissue. In other embodiments, the hepatocytes are derived from in vitro differentiation of pluripotent cells, including, but not limited to, embryonic stem cells (ESCs), induced pluripotent stem cells (iPSCs), mesenchymal stem cells (MSCs), adipose-derived stem cells (ADSCs), and oval cells. Methods for inducing pluripotent cells to differentiate in vitro into hepatocytes are known in the art. Such non-tissue derived hepatocytes are suitable for isolation and processing methods using elutriation as provided. Accordingly, in some embodiments, the provided methods and hepatocyte preparations comprise in vitro differentiated pluripotent cell-derived hepatocytes. In certain embodiments, the provided methods and hepatocyte preparations comprise in vitro differentiated ESC-derived, iPSC-derived, MSC-derived, ADSC-derived, or oval cell-derived hepatocytes.

In one embodiment, a method is provided for producing a clean preparation of isolated hepatocytes from a less clean preparation of isolated hepatocytes is described, the method comprising subjecting the less clean preparation of isolated hepatocytes to centrifugal elutriation and collecting a fraction with the clean preparation of hepatocytes, wherein the clean preparation of hepatoyctes demonstrates at least one of the following attributes as compared to the less clean preparation of isolated hepatocytes:

a. less cellular debris;
b. less cell clusters;
c. less membrane blebbing;
d. increased viability;
e. increased metabolic activity;
f. increased ability to withstand cryopreservation; or
g. increased yield of targeted cell populations.

In one embodiment, the clean preparation of isolated hepatocytes comprises an increased number of single cells as compared to the less clean preparation of isolated hepatocytes. In another embodiment, the clean preparation of isolated hepatocytes demonstrates reduced membrane damage as compared to the less clean preparation of isolated hepatocytes. Without wishing to be bound by theory, isolation of hepatocytes using an elutriation step may result in less membrane damage due to fewer damaging cell centrifugation and resuspension steps.

In one embodiment, the less clean preparation is from a single donor.

In one embodiment, the less clean preparation is from more than one donor resulting in a pooled preparation.

In one embodiment, the less clean preparation comprises a mammalian hepatocyte.

Automated cell counters cannot be effectively used with hepatocytes prepared by standard centrifugation methods due to cell size diversity within each preparation or vial of cells. The uniformity of cell size and decrease in cell clusters and clumps of the elutriated hepatocyte preparations provided herein permit the effective use of cell counters (automated and manual) to count and further assess the cells. With the decrease in cell clusters, clumps, and debris in the preparation, the elutriated hepatocyte preparations are also particularly suitable for further assessment and processing by flow cytometry instruments, such as FACS cytometers and acoustic focusing cytometers, e.g., the ATTUNE® cytometer (Life Technologies Corp.). Further, because the preparations of the instant invention can be made in the absence (or markedly less) contamination by density media (i.e. PERCOLL®), the preparations are better suited for certain applications as described herein.

The cleanliness of the hepatocyte preparation provided herein allows improvements in cell suspension processing and plating. Hepatocytes isolated with standard centrifugation methods, such as PERCOLL® centrifugation, present a significant challenge to seed in a smaller well format multi-well culture plate, such as a 96- or 384-well plate. This is mainly due to large amounts of impurities consisting of dead cells, cellular clumps and cellular debris in the standard hepatocytes preparations. In larger well formats, 6- or 24-well plates, after seeding, all impurities can be easily washed off, however, in the smaller well formats, e.g., 96- and 384-well plates, liquid tension prevents efficient washing and removal of impurities. Thus, current 96-well and 384-well plate formats seeded with hepatocytes prepared by standard methods are plagued with overwhelming amounts of dead cellular debris that may interfere with assays, including, for example, high throughput screening (HTS) assays. This is especially visible in assays requiring fluorescent imaging since any dead cell present on the plate will strongly auto-fluoresce resulting in increased background detection and thus artifactual results. Also, cell preparations containing clumps of cells mixed with single cells can result in uneven seeding and thus uneven monolayers of hepatocytes. Monolayers with large holes between hepatocytes generally result in short lived cultures.

The clean hepatocyte preparations provided herein permit the generation of multi-well plates of hepatocytes with consistency among the wells in, for example, cell number, viability, evenness of seeding, etc. With such clean hepatocyte preparations and dead cells, cellular debris, and cellular aggregates removed prior to plating, artifacts can be minimized. These overall clean hepatocyte preparations can be effectively and efficiently seeded on smaller well format multi-well plates, including, for example, 96-well, 384-well, and 1536-well plates, and other miniaturized culture surfaces as would be appreciated by those of skill in the art. These clean hepatocyte preparations provide cultures with more physiological like behavior by creating nearly confluent (e.g., 95-100%) monolayers devoid of dead cells, cellular debris, and clumps of cells. They also create cultures that can stay metabolically competent longer than cultures isolated using PERCOLL® centrifugation methods.

In one embodiment, a method is provided for making a hepatocyte monolayer in a multi-well plate, the method comprising dispensing a clean hepatocyte preparation into at least one well of the plate. In some embodiments, the clean hepatocyte preparation dispensed in the well as a single cell suspension. In some embodiments, the dispensing is automated, for example performed by a robotic device.

In one embodiment, provided is a multi-well culture surface, e.g., a plate, comprising a clean hepatocyte preparation. In one embodiment, provided is a multi-well culture plate having at least one well comprising a hepatocyte monolayer plated from a single cell preparation.

In some embodiments, provided is a 384-well culture plate having at least one well seeded with any of about 5,000, about 10,000, about 15,000, or about 20,000 to any of about 15,000, about 20,000, about 25,000, or about 30,000 hepatocytes per well. In another embodiment, provided is a 384-well culture plate having at least one well seeded with about 15,000-20,000 hepatocytes per well. In a certain embodiment, provided is a 384-well culture plate having at least 100 wells seeded with about 15,000-20,000 hepatocytes per well. In a certain embodiment, provided is a 384-well culture plate having a majority of wells seeded with about 15,000-20,000 hepatocytes per well.

In some embodiments, provided is a 1536-well culture plate having at least one well seeded with any of about 250, about 500, about 1,000, about 2000, or about 3,000 to any of about 5,000, about 6,000, about 7,000, about 7500, about 8000 or about 9,000 hepatocytes per well. In another embodiment, provided is a 1536-well culture plate having at least one well seeded with about 1,000-7,500 hepatocytes per well. In a certain embodiment, provided is a 1536-well culture plate having at least 400 wells seeded with about 1,000-7,500 hepatocytes per well. In a certain embodiment, provided is a 1536-well culture plate having a majority of wells seeded with about 1,000-7,500 hepatocytes per well.

In one embodiment, provided is a 96-well culture plate having at least one well seeded with any of about 10,000, about 15,000, about 20,000, about 25,000, about 30,000, or about 35,000 to any of about 60,000, about 65,000, about 70,000, about 75,000, about 80,000, about 85,000, or about 90,000 hepatocytes per well. In another embodiment, provided is a 96-well culture plate having at least one well seeded with about 25,000-75,000 hepatocytes per well. In a certain embodiment, provided is a 96-well culture plate having at least 30 wells seeded with about 25,000-75,000 hepatocytes per well. In a certain embodiment, provided is a 96-well culture plate having a majority of wells seeded with about 25,000-75,000 hepatocytes per well.

In some embodiments, provided is a 96-, 384-, or 1536-well culture plate having at least one well comprising a hepatocyte monolayer >80% confluent. In certain embodiments, provided is a 96-, 384-, or 1536-well culture plate having at least one well comprising a hepatocyte monolayer >85% confluent, >90% confluent, 90%-99% confluent, >95% confluent, >98% confluent, or 100% confluent. In some embodiments, provided is a 96-, 384-, or 1536-well culture plate having at least one well comprising a hepatocyte monolayer with >90% viability. In certain embodiments, provided is a 96-, 384-, or 1536-well culture plate having at least one well comprising a hepatocyte monolayer with >93% viability, >95% viability, or >97% viability.

The isolation methods provided using elutriation centrifugation produce single cell suspensions of hepatocytes which can be seeded in multi-well plates to provide a confluent cobblestone monolayer in the well. In some embodiments, provided are 96, 384-, or 1536-well culture plates comprising hepatocytes that are viable after 5-7 days in culture. In some embodiments, provided are 96-, 384-, or 1536-well culture plates having at least one well comprising a hepatocyte monolayer with >80% viability after 5-7 days in culture. In other embodiments, provided are 96-, 384-, or 1536-well culture plates having at least one well comprising a hepatocyte monolayer with >85%, >90%, or >95% viability after 5-7 days in culture.

The multi-well format plates, e.g., 96-well, 384-well, 1536-well plates, seeded with hepatocytes are suitable for HTS assays for, for example, drug development and predictive toxicology.

The elutriated hepatocyte preparations provided herein are also characterized in having an enhanced ability to attach to a culture surface, for example a plastic dish, plastic well or slide. Accordingly, in one embodiment, a clean preparation of hepatocytes is provided in which at least about 50% of the hepatocytes attach to a culture surface within about 2-6 hours of the hepatocyte suspension contacting the surface. In certain embodiments, a clean preparation of hepatocytes is provided in which at least about 60% of the hepatocytes attach to a culture surface within about 2-6 hours of the hepatocyte suspension contacting the surface. In certain embodiments, a clean preparation of hepatocytes is provided in which at least about 70% of the hepatocytes attach to a culture surface within about 2-6 hours of the hepatocyte suspension contacting the surface. In certain embodiments, at least about 80% of the hepatocytes attach to the culture surface within about 2-6 hours of the hepatocytes suspension contacting the surface. In certain embodiments, at least about 90% of the hepatocytes attach to the surface within about 2-6 hours of the hepatocytes suspension contacting the surface.

In one embodiment, a method is provided for generating a culture surface with cells attached in which the method comprises the use of a hepatocyte preparation with enhanced ability to attach to a culture surface. An exemplary method for attaching cells, such as a hepatocyte preparation, to a culture surface, includes diluting a suspension of the cells to a desired seeding density in an attachment medium. The cells at the desired density are dispensed on to the culture surface, e.g., a multi-well plate, using, for example, a pipet or pipet tip, and the culture surface with dispensed cells is incubated under conditions sufficient to allow cell attachment to the surface. In some cases, such as when a monolayer of cells is the desired outcome, the culture plate containing the dispensed cells is briefly moved with north/south and east/west motions to disperse cells prior to incubation. An exemplary incubation step for hepatocyte attachment is for 2-6 hours at 37° C. Exemplary attachment media for hepatocytes include, but are not limited to, William's Medium E (without phenol red) and William's Medium E (without phenol red) supplemented with Hepatocyte Plating Supplement Pack (Invitrogen). Other methods for handling or cultivating cells in a manner in which they will attach are known and would be understood by those of skill in the art.

The centrifugal elutriation methods provided are of use in isolating a clean preparation of hepatocytes and of use in isolating a clean preparation of a desired NPC, such as Kupffer cells and hepatic stellate cells. Such cell preparations are particularly suited for the generation of a co-culture of the hepatocyte and the NPC. For the hepatocyte/NPC co-culture, either the hepatocyte or the NPC or both are clean preparations prepared by the centrifugal elutriation methods provided. In one embodiment, a hepatocyte from a clean hepatocyte preparation is co-culture with a Kupffer cell from a clean Kupffer cell preparation. In certain embodiments, a ratio of 2:1 hepatocytes to Kupffer cell is used to generate the co-culture.

In one embodiment, the hepatocyte is selected from the group consisting of a human hepatocyte, a porcine hepatocyte, a simian hepatocyte (for example, a cynomologous, marmoset, rhesus or other monkey hepatocyte), a canine hepatocyte, a feline hepatocyte, a bovine hepatocyte, an equine hepatocyte, an ovine hepatocyte, a rabbit hepatocyte, and a rodent hepatocyte (for example, a rat or a mouse hepatocyte).

In other embodiments, the hepatocyte is a fish hepatocyte, an avian hepatocyte, a reptilian hepatocyte, or an amphibian hepatocyte.

In one embodiment, the pooled preparation comprises hepatocytes selected with respect to at least one metabolic activity.

In one embodiment, the metabolic activity is selected from the group consisting of coumarin 7-hydroxylase (COUM), dextromethorphan O-demethylase (DEX), 7-ethoxycourmarin O-deethylase (ECOD), activities responsible for the phase II metabolism of 7-hydroxycoumarin (7-HCG and 7-HCS), mephenytoin 4-hydroxylase (MEPH), testosterone 6 (beta)-hydroxylase (TEST), tolbutamide 4-hydroxylase (TOLB), phenacetin O-deethylase (PHEN), chloroxazone 6-hydroxylase (CZX), paclitaxel hydroxylase, and bupropion hydroxylase.

In one embodiment, the clean preparation exhibits at least about 50%, 60%, or 70% or greater viability.

In one embodiment, the clean preparation exhibits at least about 80%, 90% or 95% viability.

Viability may available techniques, for example, it may be measured by trypan blue exclusion. FACS analysis or flow cytometry (i.e. using propidium iodide) also provides a method of characterizing preferred populations of hepatocytes which have desired viability characteristics. For example, flow cytometry may be used to characterize the percentage of the hepatocytes in the preparation as being particularly desirable (i.e. as described in the FIGS. herein). Other functional techniques may be utilized to measure viability. Unless otherwise described, viability is measured using trypan blue exclusion.

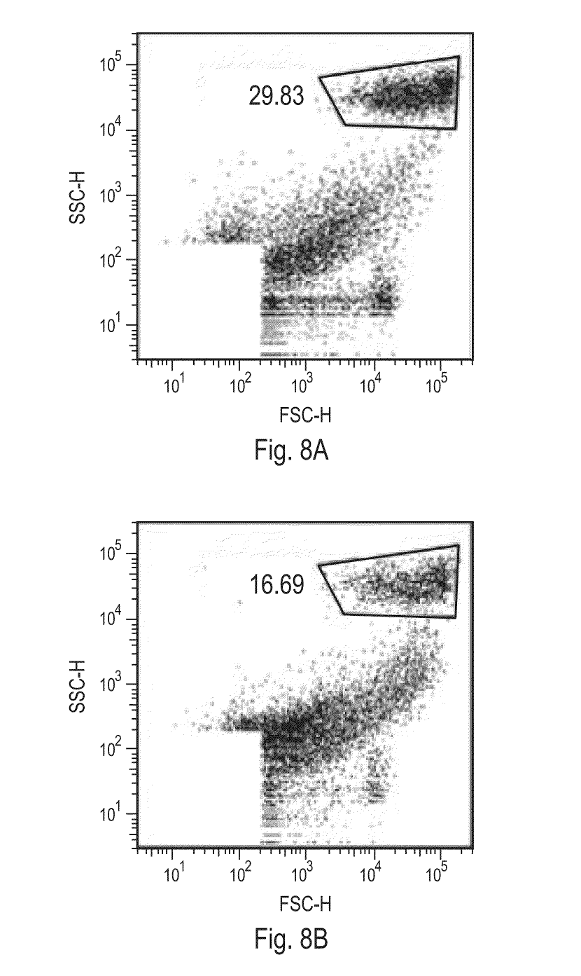

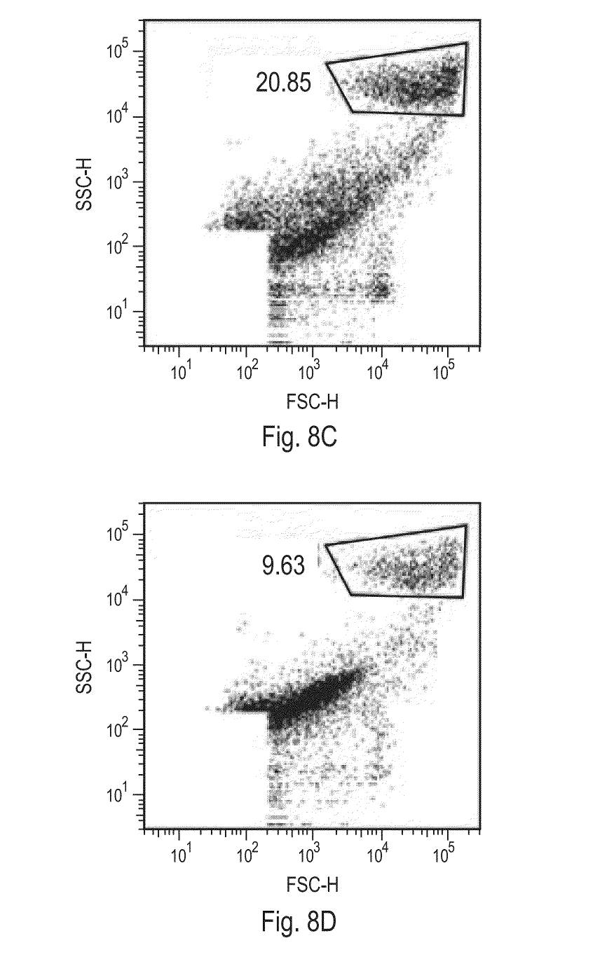

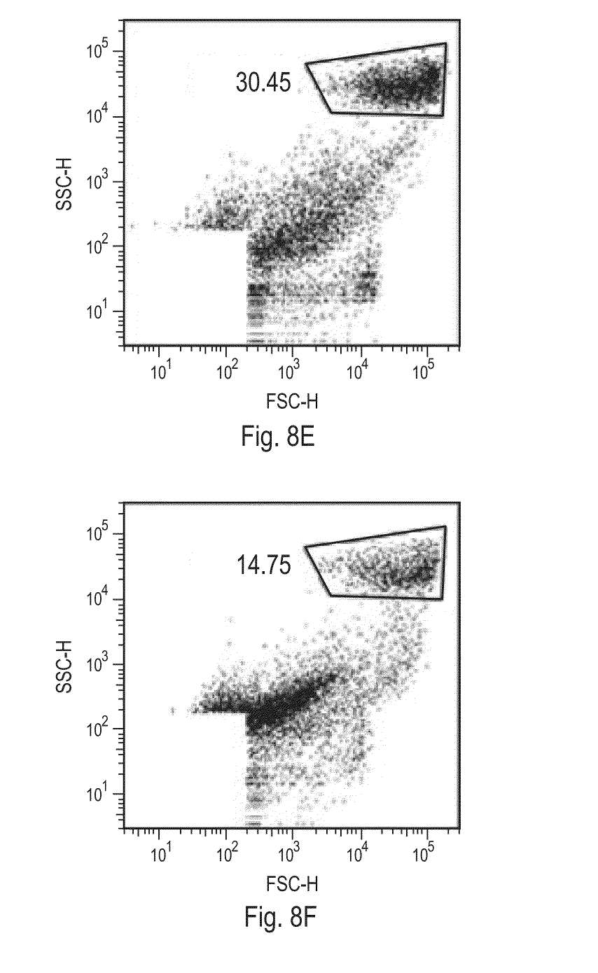

In one embodiment, the hepatocyte preparation of the instant invention achieves a score of at least 20 when subjected to flow cytometric analysis and subsequent analysis of the hepatocytes according to the methods described herein (see, for example, FIG. 8). In one embodiment, the score is at least 20.85. In one embodiment, the score is at least 25, at least 29.83, at least 30, or at least 30.45.

In one embodiment, the less clean preparation has been cryopreserved at least once.

In one embodiment, the less clean preparation has been cryopreserved at least twice.

In one embodiment, a clean preparation of hepatocytes produced by any of the method embodiments is described. Such clean hepatocyte preparations allow for more effective miniaturization of these liver-like model systems for higher density screening applications.

In one embodiment, a method is provided of investigating in vitro drug metabolism is described, the method comprising incubating a clean preparation of hepatocytes in the presence of a xenobiotic, and determining the metabolic fate of the xenobiotic, or the affect of the xenobiotic on the hepatocytes or on an enzyme or metabolic activity thereof.

In one embodiment, a preparation of hepatocytes is described, where the preparation is characterized by at least one of the following:

less than 10% of the total number of hepatocytes are present in identifiable clumps;
less than 10% of the total mass of the preparation is cellular debris;
less than 10% of the hepatocytes in the preparation exhibit membrane blebbing;
less than 10% of the hepatocytes exhibit the characteristics of Zone 1;
less than 10% of the hepatocytes exhibit the characteristics of Zone 2;
less than 10% of the hepatocytes exhibit the characteristics of Zone 3; or
the hepatocytes are substantially derived from a donor with BMI>30.

In one embodiment, a preparation of hepatocytes is described, where the preparation is characterized by at least one of the following:

less than 9% of the total number of hepatocytes are present in identifiable clumps;
less than 9% of the total mass of the preparation is cellular debris;
less than 9% of the hepatocytes in the preparation exhibit membrane blebbing
less than 9% of the hepatocytes exhibit the characteristics of Zone 1;
less than 9% of the hepatocytes exhibit the characteristics of Zone 2;
less than 9% of the hepatocytes exhibit the characteristics of Zone 3; or
greater than 50% of the hepatocytes are derived from a donor with BMI>30.

In one embodiment, a preparation of hepatocytes is described, where the preparation is characterized by at least one of the following:

less than 8% of the total number of hepatocytes are present in identifiable clumps;
less than 8% of the total mass of the preparation is cellular debris;
less than 8% of the hepatocytes in the preparation exhibit membrane blebbing
less than 8% of the hepatocytes exhibit the characteristics of Zone 1;
less than 8% of the hepatocytes exhibit the characteristics of Zone 2;
less than 8% of the hepatocytes exhibit the characteristics of Zone 3; or
greater than 70% of the hepatocytes are derived from a donor with BMI>30.

In one embodiment, a preparation of hepatocytes is described, where the preparation is characterized by at least one of the following:

less than 7% of the total number of hepatocytes are present in identifiable clumps;
less than 7% of the total mass of the preparation is cellular debris;
less than 7% of the hepatocytes in the preparation exhibit membrane blebbing
less than 7% of the hepatocytes exhibit the characteristics of Zone 1;
less than 7% of the hepatocytes exhibit the characteristics of Zone 2;
less than 7% of the hepatocytes exhibit the characteristics of Zone 3; or
greater than 75% of the hepatocytes are derived from a donor with BMI>30.

In one embodiment, a preparation of hepatocytes is described, where the preparation is characterized by at least one of the following:

less than 6% of the total number of hepatocytes are present in identifiable clumps;
less than 6% of the total mass of the preparation is cellular debris;
less than 6% of the hepatocytes in the preparation exhibit membrane blebbing;
less than 6% of the hepatocytes exhibit the characteristics of Zone 1;
less than 6% of the hepatocytes exhibit the characteristics of Zone 2;
less than 6% of the hepatocytes exhibit the characteristics of Zone 3; or
greater than 80% of the hepatocytes are derived from a donor with BMI>30.

In one embodiment, a preparation of hepatocytes is described, where the preparation is characterized by at least one of the following:

less than 5% of the total number of hepatocytes are present in identifiable clumps;
less than 5% of the total mass of the preparation is cellular debris;
less than 5% of the hepatocytes in the preparation exhibit membrane blebbing;
less than 5% of the hepatocytes exhibit the characteristics of Zone 1;
less than 5% of the hepatocytes exhibit the characteristics of Zone 2;
less than 5% of the hepatocytes exhibit the characteristics of Zone 3; or
greater than 90% of the hepatocytes are derived from a donor with BMI>30.

In one embodiment, a preparation of hepatocytes is described, where the preparation is characterized by at least one of the following:

less than 4% of the total number of hepatocytes are present in identifiable clumps;
less than 4% of the total mass of the preparation is cellular debris;
less than 4% of the hepatocytes in the preparation exhibit membrane blebbing;
less than 4% of the hepatocytes exhibit the characteristics of Zone 1;
less than 4% of the hepatocytes exhibit the characteristics of Zone 2;
less than 4% of the hepatocytes exhibit the characteristics of Zone 3; or
the hepatocytes are substantially derived from a donor with BMI>40.

In one embodiment, a preparation of hepatocytes is described, where the preparation is characterized by at least one of the following:

less than 3% of the total number of hepatocytes are present in identifiable clumps;
less than 3% of the total mass of the preparation is cellular debris;
less than 3% of the hepatocytes in the preparation exhibit membrane blebbing;
less than 3% of the hepatocytes exhibit the characteristics of Zone 1;
less than 3% of the hepatocytes exhibit the characteristics of Zone 2;
less than 3% of the hepatocytes exhibit the characteristics of Zone 3; or
greater than 70% of the hepatocytes are derived from a donor with BMI>40.

In one embodiment, a preparation of hepatocytes is described, where the preparation is characterized by at least one of the following:

less than 2% of the total number of hepatocytes are present in identifiable clumps;
less than 2% of the total mass of the preparation is cellular debris;
less than 2% of the hepatocytes in the preparation exhibit membrane blebbing;
less than 2% of the hepatocytes exhibit the characteristics of Zone 1;
less than 2% of the hepatocytes exhibit the characteristics of Zone 2;
less than 2% of the hepatocytes exhibit the characteristics of Zone 3; or
greater than 90% of the hepatocytes are derived from a donor with BMI>40.

In one embodiment, a preparation of hepatocytes is described, where the preparation is characterized by at least one of the following:

less than 1% of the total number of hepatocytes are present in identifiable clumps;
less than 1% of the total mass of the preparation is cellular debris;
less than 1% of the hepatocytes in the preparation exhibit membrane blebbing;
less than 1% of the hepatocytes exhibit the characteristics of Zone 1;
less than 1% of the hepatocytes exhibit the characteristics of Zone 2;
less than 1% of the hepatocytes exhibit the characteristics of Zone 3; or
the hepatocytes are substantially derived from a donor with BMI>50.

In one embodiment, the hepatocytes are collected at less than 1000 rpm during centrifugal elutriation.

In one embodiment, a method for separating a Zone 1 hepatocyte from a Zone 2 or Zone 3 hepatocyte is described comprising elutriating a hepatocyte composition comprising at least one Zone 1 hepatocyte and at least one hepatocyte selected from a Zone 2 hepatocyte and a Zone 3 hepatocyte.

In one embodiment, a method for separating a Zone 2 hepatocyte from a Zone 1 or Zone 3 hepatocyte is described comprising elutriating a hepatocyte composition comprising at least one Zone 2 hepatocyte and at least one hepatocyte selected from a Zone 1 hepatocyte and a Zone 3 hepatocyte.

In one embodiment, a method for separating a Zone 3 hepatocyte from a Zone 1 or Zone 2 hepatocyte is described comprising elutriating a hepatocyte composition comprising at least one Zone 3 hepatocyte and at least one hepatocyte selected from a Zone 1 hepatocyte and a Zone 2 hepatocyte.

In one embodiment, a method for isolating Zone 1 hepatocytes from Zone 2 or Zone 3 hepatocytes is described comprising (i) elutriating a hepatocyte composition comprising at least one Zone 1 hepatocyte and at least one hepatocyte selected from a Zone 2 hepatocyte and a Zone 3 hepatocyte and (ii) collecting an eluted fraction predominantly comprising Zone 1 hepatocytes.

In one embodiment, a method for isolating Zone 2 hepatocytes from Zone 1 or Zone 3 hepatocytes is described comprising (i) elutriating a hepatocyte composition comprising at least one Zone 2 hepatocyte and at least one hepatocyte selected from a Zone 1 hepatocyte and a Zone 3 hepatocyte and (ii) collecting an eluted fraction predominantly comprising Zone 2 hepatocytes.

In one embodiment, a method for isolating Zone 3 hepatocytes from Zone 1 or Zone 2 hepatocytes is described comprising (i) elutriating a hepatocyte composition comprising at least one Zone 3 hepatocyte and at least one hepatocyte selected from a Zone 1 hepatocyte and a Zone 2 hepatocyte and (ii) collecting an eluted fraction predominantly comprising Zone 3 hepatocytes.

In one embodiment the centrifugal elutriation method described herein is performed on a scale large enough for commercial applications.

As described above, the methods of the present invention are applicable to hepatocytes of human and other animals, thus allowing one to prepare hepatocytes from various animal species. Such preparations are expected to provide an alternative from and, in many cases, an improvement as compared to hepatocyte preparations of the prior art.

The following examples are provided by way of illustration and not by way of limitation.

### EXAMPLES

### Example 1

Clean elutriator (Beckman Coulter JE-5.0) by flushing first with 70% EtOH followed by flushing with H₂O. Load hepatocyte preparation into the elutriator at 1400 rpm with a flow rate of 40 ml/minute. Decrease the speed to 100 rpm for 20 minutes for a wash step. This will allow centrifugal elutriation of hepatocytes from debris, non-parenchymal cells, and dead cells. Collect hepatocytes between the range of 1000-400 rpm. Centrifuge hepatocytes. Count hepatocytes. Prepare hepatocytes for cryopreservation (if desired).

### Example 2

Hepatocyte purification by elutriation—constant flow, varied centrifugation method. (For large elutriation chamber, optimized for hepatocytes obtained from donors with BMI of <30)

Buffers: 1 L Hank's Balanced Salt Solution (HBSS) buffer Ca+/Mg+ containing 2 g Bovine Serum Albumin (BSA), 3 mg DNAse, 2.5 ml penicillin-streptomycin (P/S). Each elutriation requires approximately 4-6 L of buffer. All buffers are kept and used at 4° C.

### 1. Elutriation Preparation and Priming

Turn on the elutriator (Beckman Coulter JE-5.0) and peristaltic pump. The temperature on the elutriator should be set to 4° C. Begin to pump 500 ml of 70% alcohol for cleaning at 150 ml per minute. Once starting the pump, invert the bubble trap to collect fluid until it is about 70% full. Watch the pressure gauge carefully throughout the procedure so the pressure remains below 10-15 psi. Remove air bubbles from the elutriator chamber by manually spinning the chamber. This should be done quickly, reaching about 120 rpm, and then stopped quickly. Repeat until the chamber is filled with liquid and no air remains. When the 500 mL of 70% alcohol has finished cycling, run air through tubing and elutriator. Prepare to load 1 Liter of HBSS to flush the tubing and chamber. An additional 2-4 Liters of HBSS should be ready for the entire elutriation procedure. Repeat the flushing process by filling the air bubble trap and spinning the air out of the elutriation chamber. After the first 200 mL have cycled the elutriation system, run air and then repeat the flushing process with additional buffer or media. Close the lid of the elutriation chamber.

Keeping buffer loading at continuous 150 mL/min flow rate, start elutriator first by increasing speed form 0 to 500 rpm, wait for 2 minutes and then further increase speed up to 1400 rpm making sure that pressure gauge does not increase over 10-15 psi. When elutriator reaches centrifugation speed of 1400 rpm, decrease flow speed from 150 ml/min to 50 mL/min. At this point, elutriator is ready for loading hepatocytes.

### 2. Loading of Hepatocytes

After adjusting the Beckman Coulter JE-5.0 elutriator speed to 1400 rpm (the loading speed for hepatocytes), turn on the chamber light and adjust the chamber to the viewing position through the glass door of the lid. Use the peristaltic pump speed at 50 mL/min flow rate to load the cells. After loading the cells, decrease the elutriation speed from 1400 rpm to 1000 rpm and allow cells to wash for 15 to 20 minutes (750-1000 ml of buffer). This speed will allow the non-parenchymal cells, debris, and dead hepatocytes to be eluted while maintaining the live hepatocytes in the chamber. This fraction of cells does not need to be collected.

### 3. Collect Hepatocytes

Gradually decrease elutriation speed from 1000 to 400 rpm and collect the live fraction of hepatocytes for about 15 to 20 minutes. Hepatocytes may be collected at intermediate fractions to separate different size and density hepatocytes based on zonal distribution or fat content, or collected as one large fraction. The speed of 1000 rpm to 800 rpm will elute the small size hepatocytes, predominantly Zone 1 hepatocytes. The speed of 800 rpm to 600 rpm will elute larger size hepatocytes and some fatty cells, predominantly Zone 2 cells with some overlap with Zones 1 and 3. The speed of 600 rpm to 400 rpm will elute large bi-nucleated cells and fatty cells, predominantly Zone 3 cells. The speed of 400 rpm to 0 rpm will elute the fraction which contains the aggregated and clumps of hepatocytes. This fraction does not need to be included in the live fraction.

Approximate time of procedure: 1-2 hr, 1 person required.

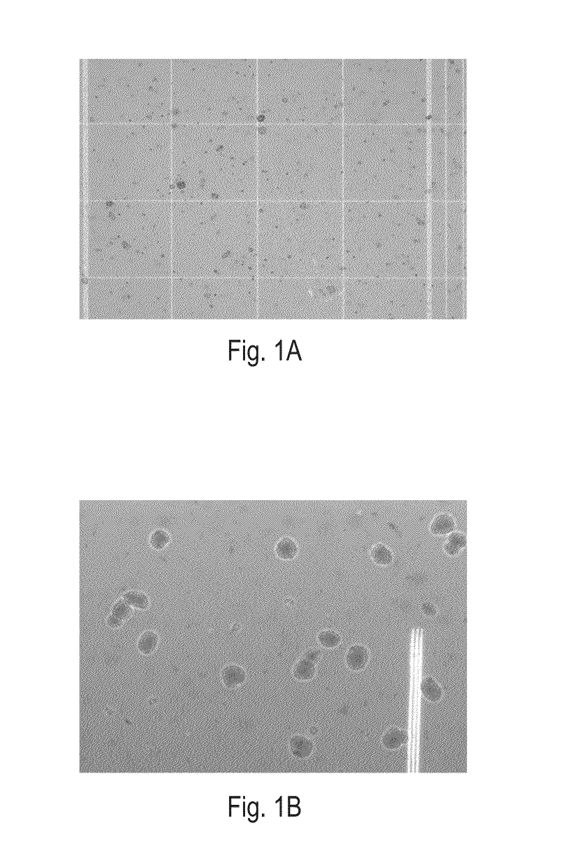

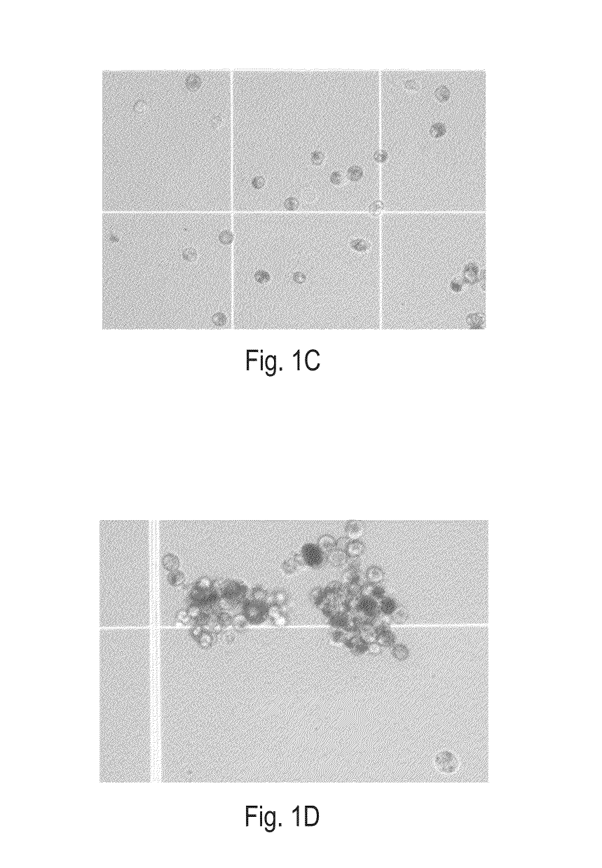

A centrifugal elutriation procedure was performed with a hepatocyte preparation as described above. A Beckman J6-M1 centrifuge containing a JE5.0 elutriator rotor was used. All centrifugation speeds were performed at a constant 50 mL/minute flow rate and fractions were collected at 1400 rpm, at 1400-1000 rpm, 1000-300 rpm, and 300-0 rpm. Samples from the eluted fractions were treated with Trypan Blue stain (Invitrogen) and viewed on a hemacytometer to assess cell viability. Representative images of the stained samples of the eluted fractions are presented in FIG. 1. Debris and nonparenchymal cells (NPCs) were collected in the 1400 rpm fraction (FIG. 1a). Dead hepatocytes (stained blue) were collected in the 1400-1000 rpm fraction (FIG. 1b). Live hepatocytes were collected as single cells in the 1000-300 rpm fraction (FIG. 1c). Clumps and aggregates of hepatocytes were collected in the 300-0 rpm fraction (FIG. 1d).

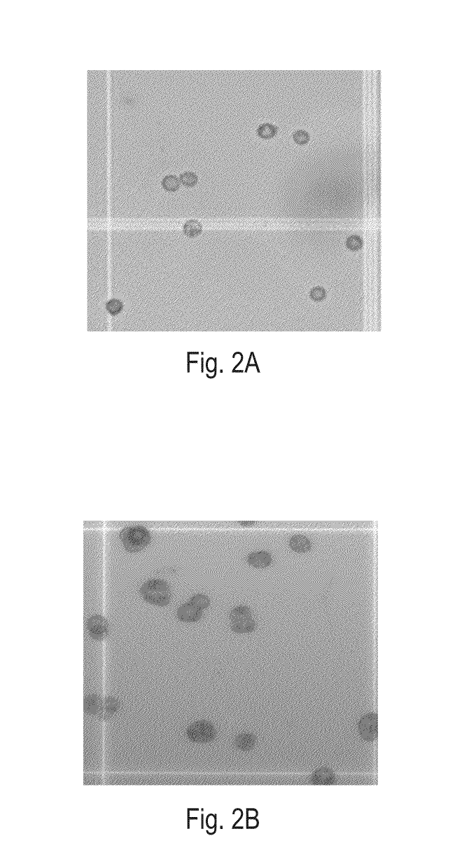

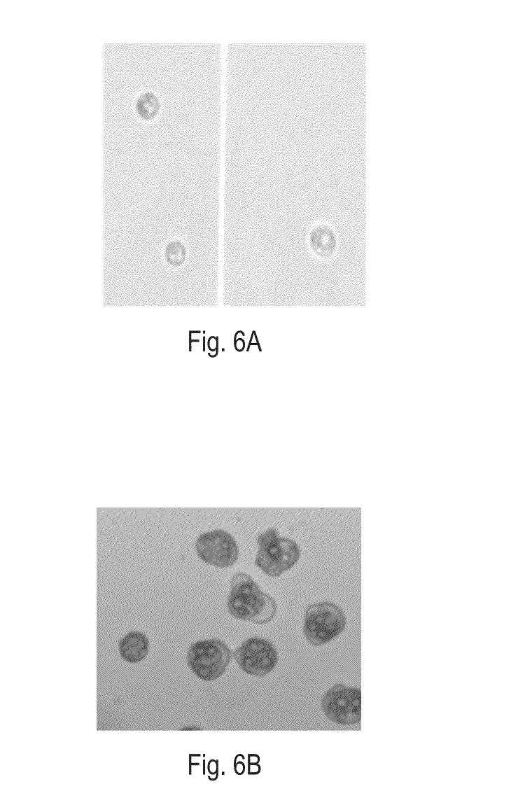

A centrifugal elutriation procedure was performed to separate a hepatocyte preparation based on the Zone(s) of origin as described above. All centrifugation speeds were performed at a constant 50 mL/minute flow rate and fractions were collected at 1000-800 rpm and 800-6000 rpm. Representative images of the stained samples of the eluted fractions are presented in FIG. 2. A smaller size population of hepatocytes collected in the 1000-800 rpm fraction (FIG. 2a) is predominantly isolated from Zones 1 or 2. A larger size population of hepatocytes collected in the 800-600 rpm fraction (FIG. 2b) is predominantly isolated from Zones 2 or 3. FIG. 6 also depicts hepatocytes isolated from Zones 1 and 3.

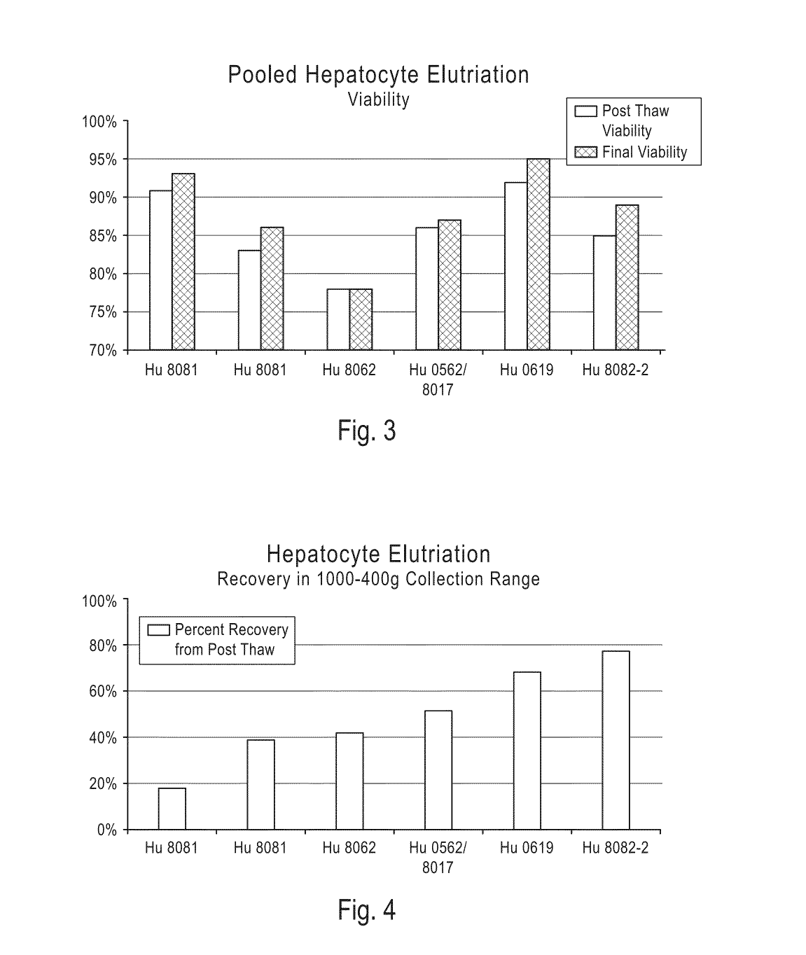

Cryopreserved human hepatocyte preparations were thawed and subjected to a centrifugal elutriation procedure. Cell viability, measured using a standard Trypan Blue exclusion assay, was determined immediately following thaw of the cryopreserved cells and following elutriation. The centrifugation speed was performed at a constant 50 mL/minute flow rate and a fraction was collected at 1000-300 rpm. As shown in FIG. 3, viability of five of the six hepatocyte preparations increased following the elutriation step and the viability of the sixth preparation (Hu 8062) was the same before and after elutriation. FIG. 4 depicts the percent recovery of hepatocytes following elutriation.

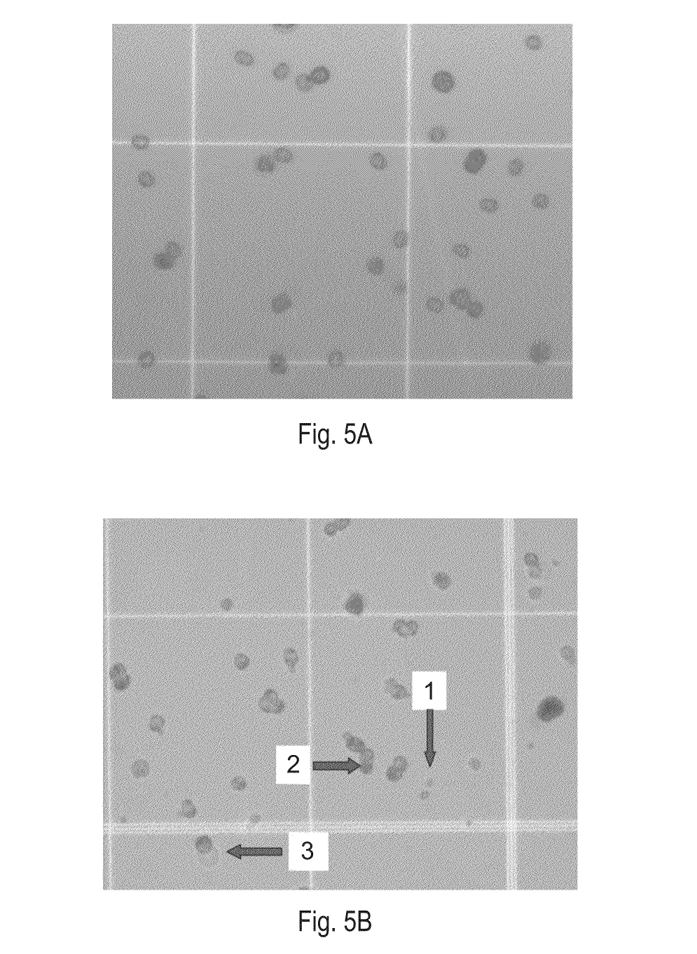

As provided herein, centrifugal elutriation yields an improved, clean preparation of hepatocytes as compared with standard isolation procedures, such as those relying on PERCOLL® centrifugation. An example of differences between such preparations of cryopreserved hepatocytes is shown in FIG. 5. The image of a sample of elutriated hepatocytes shows little evidence of debris, NPCs, cell clusters, or membrane blebbing (FIG. 5a). In contrast, the image of a sample of hepatocytes prepared with a standard isolation technique using PERCOLL® centrifugation shows evidence of debris, NPCs, cell clusters, and membrane blebbing (FIG. 5b).

Similar results are obtained when fresh hepatocytes (not previously frozen) are subjected to a centrifugal elutriation step. Following elutriation with fresh hepatocytes, there was no significant improvement over standard methods in initial hepatocyte viability as measured by Trypan Blue exclusion. However, suspensions of the elutriated fresh hepatocytes showed significantly improved microscopic morphology (e.g., membrane integrity and markedly reduced presence of debris, clumps/clusters, and NPCs) compared to hepatocyte suspensions prepared using standard centrifugation methods.

### Example 3

### Plating as a Method of Manipulating Hepatocyte Preparations

Plate hepatocytes on collagen I coated plating flasks at a volume of approximately 100-150 mL per flask. Place plating flasks in 37° C. incubator with 5% CO₂ and shake plating flasks at least 3 times back and forth and left to right. Incubate approximately 10-15 minutes. Shake plating flasks, collect hepatocyte suspension into one collection bottle on ice, rinse the flasks, and add to the collection bottle. Depending on the conditions used (i.e. temperature, time, media), this plating step may be used to remove dying or blebbing cells, dead cells, debris and/or contaminating cells. In addition, aggregates or other undesirable elements may be removed. Attached components may be visualized by microscope following removal of suspension. While not wishing to be bound by theory, cells with leaking DNA, for example, may preferentially attach during this process.

### Example 4

### Flow Cytometry of Hepatocyte Preparations

All hepatocyte samples were received cryogenically preserved and stored in liquid nitrogen (vapor phase) prior to the experiment. For the experiment the samples were thawed in a water bath at 37° C. for two three minutes. The sample was then transferred into 15 ml conical tube which contained 8 ml sorting buffer (HBSS w/o Mg²⁺/Ca²⁺, supplemented with 1% glucose, 1 mM EDTA, and 25 mM HEPES). For flow cytometry 1 ml of the diluted hepatocyte suspension was transferred to a 5 ml round bottom tube equipped with a cell strainer cap. The sample was passed through the cell strainer cap to break up cell aggregates and create a single cell suspension. Each sample was then mixed 1:1 with a 0.005 mg/ml propidium iodide (PI) solution (10 mg/ml propidium iodide stock solution (Invitrogen) was diluted 1:2000 in sorting buffer). The final cell concentration of the sample ranged from 2×10⁵ to 5×10⁵ cells/ml. The samples were kept on ice prior to flow cytometry.

Flow cytometry was performed with a FACSAria™ 2 (Becton, Dickinson & Co.) using a 100 nm nozzle at 20 psi sheet pressure. Sample excitation was performed using a 488-nm laser. For each sample, 10,000 events were counted and analyzed. All data were analyzed using the flow cytometry analysis software FlowJo (Treestar Inc.).

Hepatocyte purity analysis was performed using forward scatter and side scatter parameters (FSC-H/SSC-H). A distinct SSC_{high}/FSC_{high} population was detected and identified as hepatocyte population in accordance with previous studies (Wigg et al. (2003) Anal. Biochem. 317:19-25).

Propidium iodide staining was used to assess overall viability of the previously identified hepatocyte population. Propidium iodide is a fluorescent nucleic acid binding dye which is excluded by intact cells. Cells with compromised membranes (commonly regarded a sign of cell death), will allow uptake and DNA binding of PI which will result in increased cell associated PI fluorescence. Cell health analysis was performed by using sideward scatter versus PE-A (excitation 568 nm, measures PI fluorescence emission) parameters. Based on the PE-A channel signal, we identified distinct PI_{low} and PI_{high} populations, which were classified as live or dead hepatocyte populations respectively (data not shown).

Following elutriation with fresh hepatocytes (not previously cryopreserved), there was no significant improvement in initial hepatocyte viability as measured by Trypan Blue exclusion. However, suspensions of the elutriated fresh hepatocytes showed significantly improved microscopic morphology (e.g., membrane integrity and markedly reduced presence of debris, clumps/clusters, and nonparenchymal cells (NPCs)) compared to hepatocyte suspensions prepared using standard centrifugation methods.

Multi-donor suspension pools of cryopreserved hepatocytes were subjected to centrifugal elutriation and the live, single cell hepatocyte fraction collected. Flow cytometric analysis of these hepatocyte preparations demonstrated that the elutriated preparations consistently had less debris and fewer NPCs compared with hepatocyte preparations made with standard centrifugation methods. For example, three lots of the elutriated pooled hepatocytes tested by flow cytometry were shown to be 78%, 116%, and 106% cleaner (less debris, fewer NPCs) than hepatocyte preparations made with standard methods.

### Example 5

### Metabolic Activity of Hepatocyte Preparations

Functional metabolic activity assessments of elutriated hepatocyte preparations were performed using standard assays analyzed by LC-MS/MS. Three lots of elutriated pooled hepatocytes were assessed for CYP3A4, CYP1A2 and CYP2D6 cytochrome P450 activities.

Cells were incubated with a substrate for a set time, samples were withdrawn, the reactions stopped, and the marker metabolite assessed by LM-MS/MS. CYP3A4 activity was assessed using testosterone as a substrate and 6beta-hydroxytestosterone as the marker metabolite. CYP1A2 activity was assessed using phenacetin as a substrate and acetaminophen as the marker metabolite. CYP2D6 activity was assessed using dextromethorphan as a substrate and dextrorphan as the marker metabolite. CYP2C9 activity was assessed using diclofenac as a substrate and 4′-hydroxydiclofenac as the marker metabolite.

The suspension metabolism activities between the elutriated pooled hepatocyte preparations were comparable with historical ranges of activities of pooled hepatocyte preparations made with standard methods. For example, the elutriated pooled hepatocytes had CYP3A4 activity of 417 pmol/min/million cells, CYP1A2 activity of 49 pmol/min/million cells, and CYP2D6 activity of 44 pmol/min/million cells.

As described herein, Zone 1 of the liver lobule represents periportal hepatocytes (see, for example, FIG. 7). They are mostly single nucleated hepatocytes and usually contain little or no lipid vesicles (see, for example, FIG. 6a). They function in a higher oxygen environment and are active in oxidative metabolism, glucogenesis, and ureagenesis. Zone 3 of the liver lobule represents perivenous hepatocytes which are mostly bi-nucleated hepatocytes and can contain lipid vesicles (see, for example, FIG. 6b and FIG. 7). They function in a lower oxygen environment and are active in glycolysis, lipogenesis, and xenobiotic metabolism. Zone 2 (FIG. 7) are the midzonal centrilobular hepatocytes. They are often a mix of hepatocytes from Zones 1 and 3, but mainly similar to Zone 1 hepatocytes. They can be induced.

Zonal subpopulations of hepatocytes were prepared by centrifugal elutriation and assessed for CYP3A4, CYP1A2 and CYP2D6 cytochrome P450 activities. These results were compared with the activity of pooled hepatocytes prepared by standard methods. The cytochrome P450 enzyme activity of the Zone 3 hepatocytes was higher than the activity levels of control hepatocytes prepared with standard methods: CYP3A4 had a 42% increase over control (±0.41 in Zone 3), CYP1A2 had a 62% increase over control (±0.65 in Zone 3), and CYP2D6 had a 56% increase over control (±0.65 in Zone 3). The enzyme activity from Zone 2 hepatocytes was similar to standard method controls. The enzyme activity of Zone 1 hepatocytes was progressively lower than that of control hepatocytes prepared by standard methods: CYP3A4 had a 26% decrease under control (±0.15 in Zone 1), CYP1A2 had a 24% decrease under control (±0.19 in Zone 1), and CYP2D6 had a 41% decrease over control (±0.26 in Zone 1). Activity of CYP2C9 was relatively similar across the samples as the location of the cytochrome P450 is found to be consistently expressed at the same levels across all 3 Zones of the liver.

All publications and patents mentioned in this specification are herein incorporated by reference to the same extent as if each individual publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-15. (canceled)

16. A clean preparation of isolated hepatocytes produced from a less clean population of isolated hepatocytes according to the following method:
subjecting the less clean preparation of isolated hepatocytes to centrifugal elutriation and collecting a fraction with the clean preparation of hepatocytes, wherein the clean preparation of hepatocytes demonstrates at least one of the following attributes as compared to the less clean preparation of isolated hepatocytes:
less cellular debris;
less cell clusters;
less membrane blebbing;
increased viability;
increased metabolic activity;
increased ability to withstand cryopreservation; or
increased yield of a targeted cell population.

17. (canceled)

18. A cryopreserved preparation of pooled human hepatocytes, said cryopreserved preparation being derived from more than one human donor resulting in a pooled preparation and being characterized by at least one of the following:
less than 10% of the total number of the hepatocytes are present in identifiable clumps;
less than 10% of the total mass of the preparation is cellular debris;
less than 10% of the hepatocytes in the preparation exhibit membrane blebbing;
less than 10% of the hepatocytes exhibit the characteristics of Zone 1;
less than 10% of the hepatocytes exhibit the characteristics of Zone 2; or
less than 10% of the hepatocytes exhibit the characteristics of Zone 3.

19. The cryopreserved preparation of claim 18, wherein the preparation is substantially free of a density gradient medium.

20. (canceled)

21. The cryopreserved preparation of claim 18, wherein the hepatocytes are substantially derived from donors with BMI>30.

22-24. (canceled)

25. The cryopreserved preparation of claim 18, wherein the hepatocytes are substantially derived from donors with BMI≦30.

26-31. (canceled)

32. A preparation of human hepatocytes that have been subjected to two rounds of cryopreservation with an intervening centrifugal elutriation step, wherein the hepatocytes are further characterized by a flow cytometric analysis score of at least 20.

33-41. (canceled)

42. The preparation of claim 32, wherein said preparation is derived from more than one human donor resulting in a pooled preparation.

43. The cryopreserved preparation of claim 18, wherein the pooled human hepatocyte preparation comprises hepatocytes selected with respect to at least one metabolic activity.

44. The cryopreserved preparation of claim 18, wherein the preparation of pooled human hepatocytes has been cryopreserved at least once.

45. The cryopreserved preparation of claim 18, wherein the preparation of pooled human hepatocytes has been cryopreserved twice.
